(19) United States
(12) Patent Application Publication
(10) Pub. No.: US 2021/0137465 A1
(43) Pub. Date: May 13, 2021

(54) SCANNER SYSTEM FOR DETECTING BODY CONDITION FROM VARIOUS BODY READINGS: TEMPERATURE, SIGNALS, WAVEFORM AND BIO-PHYSIOLOGICAL CHANGES

(71) Applicant: Therresa QUACH, (US)

(72) Inventors: Bia MAC, Irvine, CA (US); Therresa QUACH, Irvine, CA (US); Lam Phuoc LE, Tan Phu District, HCM City (VN); Thi Van CAO, Huntington Beach, CA (US)

(21) Appl. No.: 17/096,404

(22) Filed: Nov. 12, 2020

Related U.S. Application Data
(60) Provisional application No. 62/934,241, filed on Nov. 12, 2019.

(51) Int. Cl. A61B 5/00; A61B 5/08; A61B 5/20; A61B 5/021; A61B 5/01

(52) U.S. Cl. CPC A61B 5/7275; A61B 5/0004; A61B 5/0015; A61B 5/45; A61B 5/08; A61B 5/40; A61B 5/742; A61B 5/41; A61B 5/20; A61B 5/02108; A61B 5/01; A61B 5/6824; A61B 5/42

(57) ABSTRACT

Data analysis is combined with physiological data to provide indications of the health of bodily systems both acute and chronic time frames. The results can confirm the uniqueness of bio-metric security. It can also enhance both sport safety, rehab and fitness goals. The compared change(s) pattern can help for remote/self health monitor as well as wellness tracking.

### BACKGROUND OF THE INVENTION

The present invention is related to the medical field, and in particular to a method and apparatus for detecting disease from physiological data.

There are many existing patents for detecting heart conditions such as heart beat rate and oxygen levels in the blood. Such inventions, however, do not address other internal organs in the body that contribute an equally important part of the overall health of the human.

Thus, there is a need for a new methodology and system for evaluating the condition of the body from temperature, signals, waveform, and bio-physiological changes by the use of smart non-invasive mobile device sensors, that provides interpretations within seconds. With the vast dynamic amount of data input on the continuously per individual as well as a selected population and general population, there is a need for a special algorithm to help the built system that can learn, contribute and predict the upcoming interruption(s) together with the live-inputs.

### DESCRIPTION OF RELATED ART

U.S. Pat. No. 8,827,906 discloses a method, system and device for measuring fingertip heart rate using motion sensors, a light source and a light detector.

U.S. Pat. No. 5,735,285 discloses using a handheld device to pick up ECG signals for heart rate. Similarly, U.S. Pat. No. 6,254,614 discloses a heart monitor via audible signal outputs.

U.S. Pat. No. 6,685,633 discloses a heart monitor that a patient can hold against the chest.

U.S. Pat. No. 6,820,057 discloses a system to acquire, record and transmit ECG data regarding the heart rate and pulse monitor.

U.S. Pat. No. 8,948,832 discloses a wearable heart rate monitor with a motion and photoplethysmographic sensor.

U.S. Pat. No. Publication 2014/0235980 discloses a method, devices and a system for measuring and monitoring heart rate and pulse.

U.S. Pat. No. 8,373,787 discloses capturing and analyzing EKG data from a user, using a smartphone.

U.S. Pat. No. 7,222,075 discloses a system, method to detect emotional states using through analysis of a user's voice.

WO Publication PCT/US2013/064041 discloses that neurological disease such as Parkinson's disease can be detected from a user's speech based on human factor cepstral coefficients (HFCC). The calculated result is based on the articulation range and articulation rate of the speech. This is only significant with medical conditions that affect the speech aspect of the user like Parkinson's disease.

U.S. Pat. No. 9,198,613 discloses an apparatus and method of diagnosing health by calculating the change of voice due to the occipital part of the brain and/or the change of the occipital bone that controls the voice, thus giving rise to voice differences. Otherwise, the brain—without the occipital portion problem—cannot detect the health problem.

The above representative prior art discloses measuring and monitoring the condition of the heart along with blood oxygen level, but is unable to evaluate the condition of other main internal organs, which play a crucial part of the overall health of the user.

### SUMMARY OF THE INVENTION

The present invention provides a method, system and device for detecting diseases of the body by analyzing temperature, waveform and bio-physiological changes. Signals are captured by biosensors (either attached to the body or wireless). These signals are processed and compared to a reference data. The results are displayed on a screen along with options to connect to local healthcare providers or vendors and can be stored online or temporarily offline for future reading. At least one input is body data. The present invention provides a method, system and device for detecting uniqueness/patterns/changes of the body by analyzing temperature, waveform and bio-physiological changes. Signals are captured by biosensors (either attached to the body or wireless). These signals are processed and compared to a live-reference data. The results are displayed on a screen along with options to secure and confirm the Bio-metric Security, Sport Safety and Rehab vs. Fitness goal maintenance and/or Remote and Self Health Management vs. Wellness monitor. The system will determine when to connect to local healthcare providers or vendors based on recommendation and can be stored online or temporarily offline for future reading. At least one input is body data.

The invention incorporates an intelligent learning algorithm that uses resultant data to ensure and enhance the accuracy of the diagnostic.

The invention provides a mobile, non-invasive method which can provide well-being interpretations within seconds. Early detection is best for preventive health and provides a beneficial approach to generate awareness for healthy living.

The present invention also provides a way of revealing the interrelationship of the different body system conditions as well as the central nervous system.

### BRIEF DESCRIPTION OF THE DRAWINGS

FIG. 1 is a flow chart of the data process according to the present invention.

FIG. 2 is a flow chart of a process according to one embodiment of the invention.

### DETAILED DESCRIPTION OF THE INVENTION

Beside the user's heart rate and pulse, oximeter (oxygen level in the blood) are vital to one health can be measure and as many prior patents.

The health of the body's systems can be determined with this invention, by analyzing captured data of the user's temperature, bio-electrical signals and/or waveforms together with physiological changes. At any time, one or more abnormalities or malfunctions of one or more body systems can affect the overall user's health that show in the temperature, bio-electrical signal, waveform, and/or in changed physiological parameters. Such body systems include: (a) the cardiovascular system/circulation system, (b) the digestive/excretory system, (c) the skeletal system, (d) the muscular system, (e) the respiratory system, (f) the nervous system, (g) the endocrine system, (h) the reproductive system, (I) the renal/urinary system, (j) the immune system, (k) the lymphatic system, and (l) the integumentary/exocrine system. The parameters may be measured by attached sensors, wireless sensors, microphones and/or cameras at locations including the tongue, eye, skin, nail, body hair, wrist, finger, neck, face, head, groin, body excretion, ankle, torso left side, torso right side and brain wave/body wave.

An EKG provides a waveform that is unique to the heart. Similarly, other body systems give out unique waveforms.

By comparing the normal parameters to one and/or more waveform from the internal organs, one can determine the systemic dis-harmonies of the overall health. For example, the digestive system includes the spleen, stomach, liver, gall bladder, etc.

The captured results are then compared by a computed algorithm with prior or reference data, and the actual differences are displayed.

After the detection of the possible conditions of the body systems, preventive measures in terms of description, recommended physical activities, nutrition and lifestyle awareness are presented.

Connection and contact information of local healthcare professionals are provided as options, as well as local vendors for health products.

Optionally, captured and calculated data may be retained to improve overall accuracy.

The user may opt in to create a person health profile on the inventor's virtual storage server for open collaboration to learn from and share with a community of peers and care professionals.

In as much as the invention is subject to variations and modifications, it is intended that the foregoing shall be interpreted as merely illustrative of the invention defined by the following claims.

We claim:

1. A diagnostic method comprising:
capturing input data including a body sample and at least one other input indicative of a bio-physiological parameter or waveform,
calculating with reference to a live data base, from said body sample and said at least one other input, results indicative of or confirming a disorder or malfunction or change(s) of at least one organ or body system, and
displaying results of said calculating on a screen.

2. The method of claim 1, wherein said parameter is measured at locations of the body including at least one of the tongue, eye, skin, nail, body hair, wrist, finger, neck, face, head, groin, body excretion, ankle, torso left side, torso right side and brain wave/body wave.

3. The method of claim 1, wherein said parameters are measured by devices selected from the group consisting of attached sensors, wireless sensors, microphones and cameras.

4. The method of claim 1, further comprising a step of adjusting capacities for picking up signals for at least 1-30 seconds time frame and the focused area within ideal proximity with adjusting visual cues.

5. The method in claim 1, wherein said body systems are selected from the group consisting of:
(a) cardiovascular system/circulation system
(b) digestive/excretory system
(c) skeletal system
(d) muscular system
(e) respiratory system
(f) nervous system
(g) endocrine system
(h) reproductive system
(I) renal/urinary system
(j) immune system
(k) lymphatic system
(l) integumentary/exocrine system.

6. The method of claim 1, wherein captured data is stored to ensure and enhance diagnostics.

7. The method of claim 1, where the condition of the body systems is continuously monitored for the purpose of stabilizing vital signs and warning of possible failure.

8. The method of claim 1, wherein at least one said other input is selected from the group consisting of pulse data, oximeter data, temperature data, eye data, iridology chart, self-picture, tongue data, skin data, body hair data, body excretion data, brain wave data, body wave data, and head data.

9. The method of claim 8, wherein said oximeter data is obtained by processing a video recording of the wrist.

10. A diagnostic system comprising
a microphone for obtaining a sample of a subject's signal,
at least one other sensor for obtaining input data representative of a physical parameter or waveform of the body,
a processor for calculating, from said sample and said at input data, results indicative of a disorder or malfunction of at least one organ or body system, and
a screen for displaying results of said calculating.

11. The system of claim 10, wherein said sensors are placed on the body at locations including at least one of the eye, tongue, nail, body hair, body excretion, wrist, finger, neck, face, groin, ankle, torso left side, torso right side.

12. The system of claim 10, wherein said at least one other sensor is selected from the group consisting of attached sensors, wireless sensors, microphones and cameras.

13. The system of claim 10, further comprising means for storing at least one of said sample, said input data and said calculated results.

14. The system of claim 10, wherein said processor calculates, from said sample and said at input data, results indicative of BPM, Oxygen, Pulse, Acute and Chronic condition(s, and said screen displays results of said calculating.

15. The system of claim 10, wherein said processor for calculating, from said sample and said at input data, results will be store and produce reports per specific request.

16. The system of claim 10, wherein said processor calculates, from said sample and said at input data, results indicative of a disorder or malfunction of at least one organ or body system and Recovery Heart Rate, Effectiveness of Exercise/Activities, Oxygen, Pulse, Acute and Chronic Condition(s), and said screen displays results of said calculating of Recovery Heart Rate, Effectiveness of Exercise/Activities, Oxygen, Pulse, Acute and Chronic Condition(s).

17. The system of claim 10, wherein said processor calculates, from said sample and said at input data, results indicative of the severity of said disorder or malfunction of at least one organ or body system, and said screen displays the results of said calculating of the severity/degree of malfunction conditions.

18. The system of claim 10, wherein said processor calculates, from said sample and said input data, results indicative of a location of the disorder or malfunction of at least one organ or body system, and said screen displays results of said calculating of the location of the malfunction condition.

19. The system of claim 10, wherein said processor calculates, from said sample and said input data, results indicative of a disorder or malfunction of at least one organ or body system, and said screen displays results of said calculating as well as the suggestions for wellness preventive health, exercise, lifestyle, supplements and or seeking professional help.

20. The system of claim 10, wherein said processor calculates, from said sample and said input data, results indicating pattern(s) or change(s) or prediction of at least one organ or body system, condition(s) and said screen displays said results of said calculating as well as suggestions from alert(s), wellness preventive health, exercise, lifestyle, supplements and or seeking professional help.

21. The system of claim 10, where said processor calculates, from said sample and an average of said input data, results indicative of a uniqueness pattern/change(s)/disorder(s) or malfunction of at least one organ or body system, and
said screen displays the results of said calculating.
